(19) United States
(12) Patent Application Publication
(10) Pub. No.: US 2008/0269640 A1
(43) Pub. Date: Oct. 30, 2008

(54) Appliance for Recording Diagnostic Values in the Body

(75) Inventors: Thomas Bayer, Igersheim (DE); Roman Stauch, Assamstadt (DE)

Correspondence Address: BACHMAN & LAPOINTE, P.C., 900 CHAPEL STREET, SUITE 1201, NEW HAVEN, CT, 06510, US

(73) Assignee: WITTENSTEIN AG, Igersheim (DE)

(21) Appl. No.: 12/093,353

(22) PCT Filed: Oct. 26, 2006
(86) PCT No.: PCT/EP06/10327

§ 371 Date: May 12, 2008

(30) Foreign Application Priority Data
Nov. 17, 2005 (DE) 10 2005 055 398.2

(51) Int. Cl. A61B 5/00

(52) U.S. Cl. 600/583

(57) ABSTRACT

In an appliance for recording diagnostic values in the body, with at least one sensor element that is assigned to a housing, the housing is intended to be able to be implanted with the at least one sensor element.

### BACKGROUND OF THE INVENTION

The present invention relates to an appliance for recording diagnostic values in the body, using at least one sensor element which has an associated housing, and the housing can be implanted together with the at least one sensor element.

Appliances such as these, in particular blood glucose measurement appliances, are known on the market in many forms and versions, and are commercially available. In this case, modern methods and appliances for carrying out the methods are known, with a glucose concentration being determined outside laboratories, for example by means of test strips. In this case, the result of a chemical reaction to a substance on the strip and the blood applied to it is recorded electrically and/or photometrically. In order to repeat the measurement, a new test strip must in each case be inserted in a measurement appliance, in which case sufficient blood must be available for this purpose. Furthermore, a conventional test strip must be in a good state and must match the type of measurement appliance.

This has the disadvantage that a test method such as this allows only costly measurement because of the large number of test strips, causing pain to the patient because of the permanent need to take blood repeatedly and with the patient being bound to a specific measurement appliance and test strip. Furthermore, a measurement such as this may not be possible sufficiently frequently, for example only five times per day. In particular, tests such as these of certain patient groups, such as children, the blind, and those with physical and psychological impediments, cannot be considered reasonable in all cases. Furthermore, such test results are inaccurate, because of the limited number of measurements, in terms of changes in the blood glucose content, so that exact medication is virtually impossible.

WO 03/055391 A1 discloses a housing which can be implanted and has an integrated fixed sensor element, which is released for the purpose of a measurement. The release process generally injures connective tissue. For this purpose, a separate cover, slide or the like is provided on the housing of the apparatus in order to remove contamination from the permanently installed sensor element.

WO 03/034902 A2 discloses a sensor element which can be implanted subcutaneously. WO 01/80728 A1 describes an apparatus for automatically determining the glucose content of the blood, with an assembly which can be implanted and a multiplicity of microchambers being provided, and in which case the individual chambers can be opened and closed as required.

WO 01/03572 A1 discloses an apparatus for measuring the human glucose level, in which a catheter is used. In addition, the measurement point has a separate cleaning apparatus, in order to remove tissue parts which have been deposited from the blood.

The present invention is therefore based on the object of providing an appliance for recording diagnostic values in the body, in particular of the blood, which overcomes the stated disadvantages and in which a selected diagnostic value of the body, in particular of the blood, can be determined permanently or at desired time intervals, without causing pain to the patient, as well as exactly and precisely.

### SUMMARY OF THE INVENTION

This object is achieved by the features of the present invention, wherein it has been particularly advantageous by varying the surface area, volume or extent of an appliance which can be implanted completely, subcutaneously under the skin, fixed or implanted in a bone, to tear open or to tear encapsulation of binding tissue, so that fresh tissue fluid, in particular blood, enters the area of a sensor element.

In this case, mechanically movable elements, such as pins, needles, canulars, blades or the like, can also be moved with respect to the appliance in order, after encapsulation of a subcutaneously implanted appliance, to apply fresh tissue fluid to a sensor element. The sensor element is preferably associated with this movable element. However, it may also be arranged in the area of the housing or the housing surface. The invention is not intended to be restricted to this.

This allows diagnostic values of the body to be recorded at selectable time intervals or continuously. Considerably more measured values, for example of the glucose level, can be achieved at relatively short time intervals. The precision of the measurements can be considerably increased and the correspondingly discontinuous or continual dosage of medicaments can be subjected to appropriately exact open-loop and closed-loop control. This makes it possible to avoid hypoglycemia and hyperglycemia, while considerably improving safety and life expectancy of diabetics. Furthermore, this makes it much simpler for the patient to handle his illness, his quality of life is directly improved, and his entire health system is in this way relieved of major problems, since the handling of diabetes is considerably simplified. Furthermore, the exact determination of the blood glucose level allows a permanent supply of insulin, or the use of insulin pumps, to be subjected to closed-loop and open-loop control, so that, for example, insulin pump therapy can be carried out and automated completely independently of the patient. Furthermore, another aim is also to use the sensor element to additionally record and evaluate further values, such as the heart rate, blood pressure, vitamin levels, and illness causes. This is likewise intended to be within the scope of the present invention.

One aim of the present invention is to determine any desired values, diagnostic values or blood values permanently, continually or at selectable time intervals by means of an appliance which can be implanted completely. This is intended to allow a controlled supply of medicaments to be provided by determining, for example cholesterol values, hemoglobin values, and values of medicaments in the blood, as a concentration. Another aim in this case is also to allow, for example, the heart rate or blood pressure as well as a value of a specific medicament in the blood to be determined in order to provide closed-loop and open-loop control for an additional supply if the concentration changes.

By way of example, for pain therapy, the medicament concentration of the analgesic in the blood can be determined permanently or at selectable time intervals and, if the values change, more medicament can be given in order to achieve the required concentration in an automated form or externally again, by means of the fully implanted appliance.

The determined measured values are transmitted telemetrically and/or without contact to an external medicament supply device, or to a medicament supply device which can be implanted completely, in particular an insulin pump. This is then operated appropriately for the required concentrations or medicaments in the body. Another aim within the scope of the present invention is that it should be possible to combine the measurement system for determination of the blood glucose level and the medicament supply device or insulin pump within one system. This system can also be implanted completely.

### BRIEF DESCRIPTION OF THE DRAWINGS

Further advantages, features and details of the invention will become evident from the following description of preferred exemplary embodiments and from the drawing, in which:

FIG. 1a shows a schematically illustrated longitudinal section through an appliance for determining the blood glucose level, in a retracted in-use position;

FIG. 1b shows a schematically illustrated longitudinal section through the appliance shown in figure FIG. 1a, in an extended in-use position;

FIGS. 2a and 2b each show two further exemplary embodiments of appliances with a variable volume extent for determining the blood glucose level;

FIG. 3 shows a side view of a further exemplary embodiment of a further appliance for determining the blood glucose level adjacent to a bone;

FIG. 4 shows a schematic illustration of a further exemplary embodiment of an appliance for determining the blood glucose level as shown in FIG. 3;

FIGS. 5a and 5b show further exemplary embodiments of appliances for determining the blood glucose level.

### DETAILED DESCRIPTION

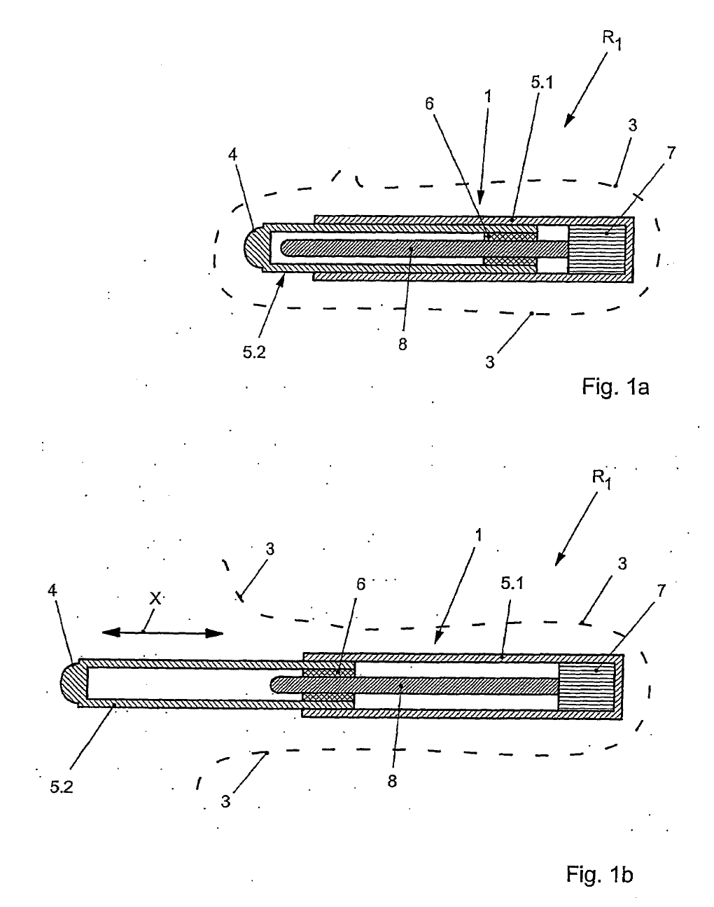

As shown in FIGS. 1a and 1b, an appliance R₁ according to the invention for determining the blood glucose level has a housing 1 which can be implanted subcutaneously under the skin. In this case, the housing 1 can be fixed adjacent to any desired bone 2, for example as is indicated in FIG. 3, or can even be inserted into a tubular bone. The invention is not intended to be restricted to this.

With the present invention, it has been found to be particularly advantageous to vary the volume, the extent or the surface area of the housing 1. Variation of the volume, extent or surface area, in particular enlargement of the housing 1, allows a connective tissue 3 to be torn into, torn or torn open, which is formed automatically over the course of time around a foreign body which has been inserted subcutaneously under the skin. The housing 1 of the appliance R₁ and therefore the sensor element 4, is no longer accessible to the tissue liquid, because of the corresponding encapsulation by means of the binding tissue 3.

FIG. 1b indicates the connective tissue 3 being torn open so that tissue fluid, in particular blood, can enter the area of the housing 1 again. Immediately after the tissue fluid enters the housing 1, the blood glucose level is determined, for example, by means of at least one sensor element 4, which is preferably in the form of a glucose measurement sensor.

The appliance R₁ essentially comprises two housing parts 5.1, 5.2 which are positioned coaxially one inside the other and can be moved backwards and forwards in the direction of the illustrated double-headed arrow X by means of at least one actuator 6, in particular a linear actuator, and a motor and/or gearbox and/or control unit 7. The sensor element 4 is preferably situated on the movable housing part 5.2. The sensor element 4 may also be arranged in the housing 1, in which case blood is taken from outside the housing 1 by means of any desired device, for example a canular, and is supplied to the sensor element 4 that is arranged in the housing 1.

The motor unit and/or gearbox unit and/or control unit 7 is supplied with power with or without the use of wires, for example inductively, and is also used for bidirectional telemetric data interchange relating to the determination of the blood glucose level.

In this case, the housing part 5.2 is moved out of the housing part 1.2 by means of the actuator 6 and/or a spindle 8, with the spindle 8 being driven via the motor unit and/or gearbox unit and/or control unit 7, and with the housing part 5.2 being moved controllably out of the housing part 5.1. This makes it possible to tear open, burst or tear any connective tissue 3 that may exist, for example as is indicated in FIG. 1b, so that tissue fluid, in particular blood, can enter the area of the sensor element 4, where the blood glucose level can be determined exactly.

This process can be repeated selectively or uncontrollably, that is to say the volume, the extent or the surface area of the housing is changed before each determination of the diagnostic values, for example before determining the blood glucose level.

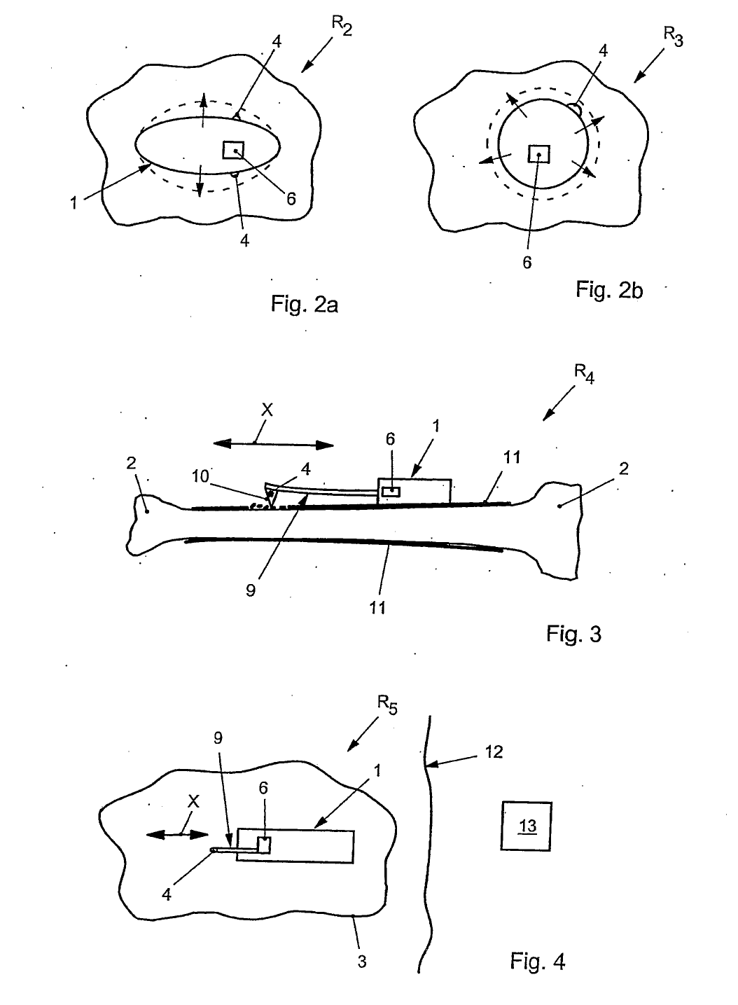

A further exemplary embodiment of an appliance R₂ is shown in FIG. 2a, in which, for example by means of an actuator 6, the volume of the housing 1 can be varied by pumping, movement, mechanical positioning or the like, in order to tear or to tear open the connective tissue 3 so that tissue fluid enters the area of the sensor element 4, as mentioned above, with the diagnostic values, such as the blood glucose level, being determined immediately after tearing open and ingress of tissue fluid.

In this case, the housing 1 may have many different cross sections, so that the invention is not restricted to one specific cross section. As is illustrated in the exemplary embodiment shown in FIG. 2b, a housing may also have a round, polygonal or platelike or similar cross section, and it may have at least one actuator 6 in order to vary the external shape, the volume or the extent for tearing into, tearing open or tearing the connective tissue.

Another aim of the present invention, as is indicated for an appliance R₄ in a further exemplary embodiment shown in FIG. 3, is for a housing 1 to be permanently connected to a bone 2, with an element 9 projecting out of the housing 1, which element 9 can be moved backwards and forwards in the direction of the illustrated double-headed X by means of at least one actuator 6, with a point 10 being provided on the element 9, which point 10 scratches the polyostium 11 of the bone, so that tissue fluid or blood then emerges and then immediately comes into contact with the sensor element 4 which is fitted to the point 10, in order to determine the diagnostic values, such as the blood glucose level. In this case, the corresponding at least one sensor element 4 may also be arranged on other areas of the element 9.

In another exemplary embodiment of the present invention as shown in FIG. 4, an appliance R₅ is illustrated in which the element 9 projects out of the housing 1, at the end of which element 9 the at least one sensor element 4 is preferably arranged.

The appliance R₅ is implanted subcutaneously under a person's skin 12, with encapsulation of the appliance R₅ by the connective tissue 3 being torn open, torn or otherwise perforated by movement of the element 9 by means of the actuator 6 in the illustrated X direction and/or by radial movement, in order then to appropriately exactly determine the glucose content of the blood by means of the sensor element 4.

This process can be repeated permanently or at selectable time intervals, for example every hour, so that the data or the exact determination of the blood glucose content is then passed with or without the use of wires to an evaluation unit 13 which then, for example, determines an insulin concentration and then appropriately controls the insulin supply.

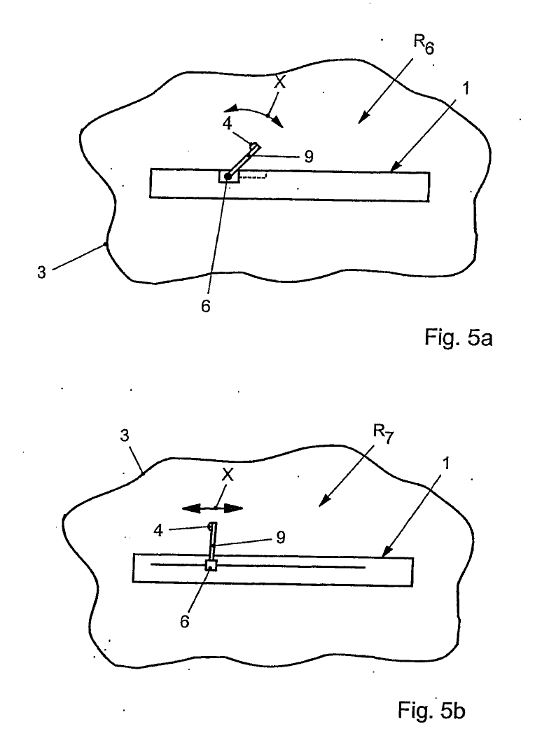

The exemplary embodiment in FIGS. 5a and 5b shows further appliances R₆ and R₇, in the case of which corresponding elements 9 can be pivoted or moved linearly in order to scratch, to penetrate or to destroy connective tissue in order then to determine the diagnostic values, such as the blood glucose level, by means of a sensor element 4 which is preferably arranged there. In this case, appropriate actuators 6, linear drives or the like, which are only indicated here, may be provided in order to move the elements 9 in each case.

The scope of the present invention is also intended to cover, for example, the at least one sensor element 4 being arranged externally on the housing 1.

1-19. (canceled)

20. An appliance for recording diagnostic values in the body, comprising at least one sensor element which has an associated housing, wherein the housing has the at least one sensor element implanted in it.

21. An appliance for recording diagnostic values in the body, comprising at least one sensor element which has an associated housing, wherein, in order to break open and/or to tear connective tissue with an implanted housing, the housing and/or the sensor element and/or a further element which is associated with the sensor element is designed to have a variable volume, extent and/or a variable surface area.

22. A housing for recording diagnostic values in the body comprising at least one sensor element which has an associated housing, wherein, in order to break open and/or to tear connective tissue with an implanted housing and with at least one sensor element, the at least one sensor element and/or an additional element can be moved with respect to the housing.

23. The appliance as claimed in at least one of claims 20 to 22, wherein, before recording a diagnostic value of the body, in particular of the blood, the volume, extent and/or surface area of the housing can be varied.

24. The appliance as claimed in claim 23, wherein the at least one sensor element and/or the at least one element is associated with the housing so that it can be moved, in particularly such that it can be moved linearly and/or can be pivoted.

25. The appliance as claimed in claim 23, wherein the at least one sensor element and/or at least one element can be moved with respect to the connective tissue with respect to the housing by means of at least one actuator immediately before recording of diagnostic values of the body, in particular of the blood, and the connective tissue can be torn open or torn in order to allow bodily fluid to come close to the area of the sensor element in order to determine the glucose or blood-sugar level.

26. The appliance as claimed in claim 23, wherein the housing is formed from at least two housing parts which are positioned coaxially one inside the other.

27. The appliance as claimed in claim 26, wherein at least one housing part has at least one associated sensor element for recording diagnostic values of the body, in particular of the blood.

28. The appliance as claimed in claim 26, wherein the housing parts which are positioned coaxially one inside the other can be moved linearly with respect to one another and/or can be rotated radially with respect to one another by means of at least one actuator, in particular a linear actuator.

29. The appliance as claimed in claim 26, wherein the housing parts are formed from a biocompatible material, or are coated with this material.

30. The appliance as claimed in claim 26, wherein a motor unit and/or a gearbox unit and/or a control unit with a spindle connected to them or it are or is provided in one housing part, with the second housing part engaging with the spindle in order to move them one inside the other or out of each other, linearly.

31. The appliance as claimed in claim 23, wherein the at least one element can be moved linearly or can be pivoted with respect to the housing and with respect to the connective tissue, in order to tear it or tear it open, by means of at least one actuator.

32. The appliance as claimed in claim 23, wherein the sensor element is in the form of a glucose measurement sensor or a sensor element for recording body-specific or blood-specific values.

33. The appliance as claimed in claim 23, wherein the element is in the form of a blade, scratcher, scraper, nail, canular or the like, which can be moved with respect to the housing in order to at least partially penetrate into the connective tissue before recording diagnostic values of the body, in particular of the blood.

34. The appliance as claimed in claim 23, wherein the element which can be moved with respect to the housing has at least one associated sensor element for recording of diagnostic values of the body, in particular of the blood.

35. The appliance as claimed in claim 23, wherein the at least one sensor element is connected to a control unit and, after recording the diagnostic value of the body, in particular of the blood, the information can be passed on with or without the use of wires to an evaluation unit and/or to a medicament supply device, in order to emit medicaments to the body, after the values have been determined.

36. The appliance as claimed in claim 23, wherein the housing can be implanted subcutaneously or can be fixed to a bone.

37. The appliance as claimed in claim 23, wherein at least one element, possibly with an integrated or fitted sensor element, in particular in the form of a scraper, can be actively controlled and moved with respect to a periostium before the recording of diagnostic values of the body, in particular of the blood, in order to tear these and to obtain tissue fluid for exact recording of diagnostic values of the body, in particular of the blood.

38. The appliance as claimed in claim 23, wherein the housing can be implanted with an integrated or connected evaluation unit and/or a medicament supply device which is separate or is integrated in the housing with the medicament being supplied subcutaneously, and with the medicaments being dispensed or emitted in a selectable dose to the body at intervals which can be connected or can be determined as required.
